(19) United States
(12) Patent Application Publication
(10) Pub. No.: US 2019/0158707 A1
(43) Pub. Date: May 23, 2019

(54) IMAGE PROCESSING DEVICE AND ELECTRONIC ENDOSCOPE SYSTEM

(71) Applicant: HOYA CORPORATION, Tokyo (JP)

(72) Inventor: Fumika YOKOUCHI, Tokyo (JP)

(73) Assignee: HOYA CORPORATION, Tokyo (JP)

(21) Appl. No.: 16/095,014

(22) PCT Filed: Jul. 25, 2017
(86) PCT No.: PCT/IB2017/054490

§ 371 Date: Oct. 19, 2018

(30) Foreign Application Priority Data
Jul. 4, 2016 (JP) 2016-132897

(51) Int. Cl. H04N 5/208; A61B 1/045; A61B 1/00

(52) U.S. Cl. CPC H04N 5/208; A61B 1/00009; A61B 1/045

(57) ABSTRACT

An image processing device includes: a plurality of sharpening circuits for enhancing different frequency components for an original image; an adjusting means for adjusting the signal level ratio of an image sharpened by each of the plurality of sharpening circuits; and a generating means for generating an enhanced image by adding up signals of images subjected to the signal level ratio adjustment at a predetermined ratio.

### TECHNICAL FIELD

The present invention relates to an image processing device and an electronic endoscope system.

### BACKGROUND ART

An image processing device that processes an original image to generate an enhanced image in which the contour (edge) has been enhanced. For example, JP 2002-183727 A (hereinafter referred to as “Patent Document 1”) describes a specific con ration of an image processing device of this type.

The image processing device described in Patent Document 1 is a device specialized in performing edge enhancement of a radiographic image. In order to obtain a radiographic image edge-enhanced by magnifying the light-dark difference, the image processing device described in Patent Document 1 employs the processing of subtracting a non-sharp image signal from an original image signal thereby performing contour definition.

### SUMMARY OF INVENTION

For example, in order to allow the operator to easily observe a body tissue in a body cavity, it is considered to enhance and display an image shot in an electronic scope using the processing illustrated in Patent Document 1. However, in the processing illustrated in Patent Document 1, the enhancing processing is available only for specific frequency components in the original image (shot image). Therefore, a problem is pointed out that, with the processing illustrated in Patent Document 1, the enhancing processing not available depending on the site in the body cavity

In view of the above problem, an objective of the present invention is providing an image processing device and an electronic endoscope system capable of applying the enhancing processing to any site in the body cavity.

The image processing device according to an embodiment of the present invention includes: a plurality of sharpening circuits for enhancing different frequency components for an original image; an adjusting means for adjusting the signal level ratio of an image sharpened by each of the plurality of sharpening circuits; and a generating means for generating an enhanced image by adding up signals of images subjected to the signal level ratio adjustment at a predetermined ratio.

The image processing device according to an embodiment of the present invention may include a means for increasing the granularity of the original image upstream of the plurality of sharpening circuits.

The image processing device according to an embodiment of the present invention may include a means for adding up a signal of the original image and a signal of the enhanced image generated by the generating means.

The image processing device according to an embodiment of the present invention may include a clipping means for clipping the signal of each image subjected to the signal level ratio adjustment by the adjusting means.

In an embodiment of the present invention, the plurality of sharpening circuits include a set of a low-pass filter and a subtraction circuit that subtracts an output signal of the low-pass filter from the signal of the original image, and a Laplacian filter, for example.

An electronic endoscope system according to an embodiment of the present invention includes: an electronic scope; and any of the above image processing devices that processes image data shot in the electronic scope as the original image.

According to an embodiment of the present invention, an image processing device and an electronic endoscope system capable of applying the enhancing processing to any site in the body cavity is provided.

### BRIEF DESCRIPTION OF DRAWINGS

FIG. 1 is a block diagram showing a configuration of an electronic endoscope system according to an embodiment of the present invention.

FIG. 2 is a view showing a configuration of an edge enhancing circuit provided in a processor according to an embodiment of the present invention.

FIG. 3 is a graph showing the relationship between frequency components and the modulation transfer function (MTF) in an enhanced image output to a monitor in an embodiment, of the present invention.

FIG. 4 is a view showing a configuration of an edge enhancing circuit according to another embodiment of the present invention.

### DESCRIPTION OF EMBODIMENTS

Embodiments of the present invention will be described hereinafter with reference to the accompanying drawings. Note that, as an embodiment of the present invention, an electronic endoscope system will be taken as an example and described hereinafter.

[Configuration of Electronic Endoscope System 1]

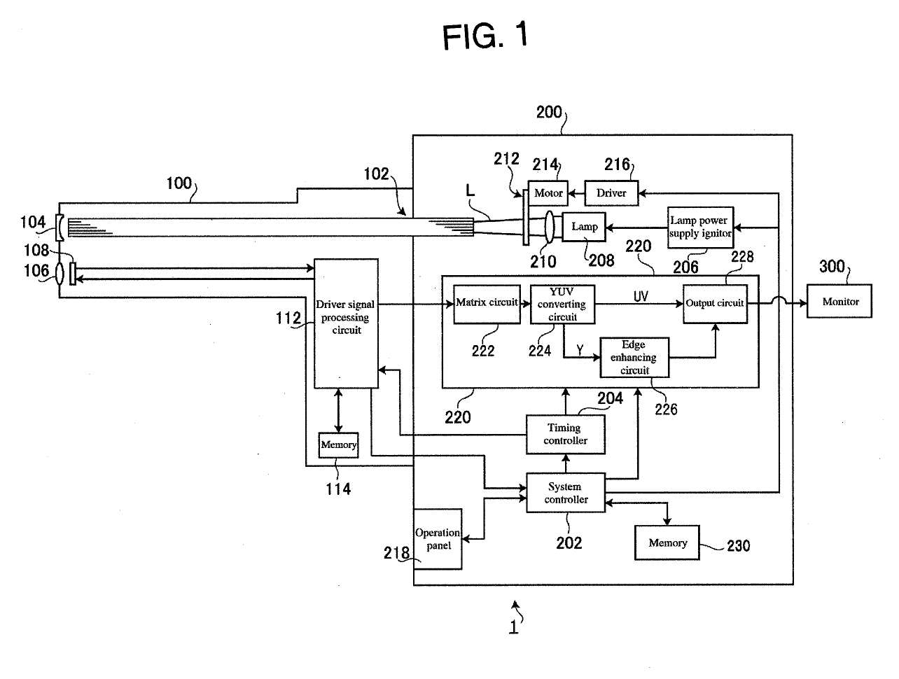

FIG. 1 is a block diagram showing a configuration of an electronic endoscope system 1 according to an embodiment of the present invention. As shown in FIG. 1, the electronic endoscope system 1 is a system specialized for medical use and includes an electronic scope 100, a processor 200, and a monitor 300.

The processor 200 includes a system controller 202 and a timing controller 204. The system controller 202 executes various programs stored in a memory 30 and controls the entire electronic endoscope system 1 in an integrated manner. Also, the system controller 202 is connected to an operation panel 218. In response to instructions from the operator input via the operation panel 218, the system controller 202 executes operation of the electronic endoscope system 1 and changes parameters for the operations. An example of the input instructions by the operator includes an instruction of switching among operation modes of the electronic endoscope system In this embodiment, as the operation modes, there are a low-frequency enhancing mode, an intermediate-frequency enhancing mode, a high-frequency enhancing mode, etc. The timing controller 204 outputs clock pulses for adjusting the timing of the operations of the parts to the circuits in the electronic endoscope system 1.

A lamp 208 radiates white light L after startup with a lamp power supply ignitor 206. The lamp 208 may be a high-intensity lamp such as a xenon lamp, a halogen lamp, a mercury lamp, and a metal halide lamp or a light emitting diode (LED), for example. The white light L radiated from the lamp 208 is restricted to a proper light amount via a diaphragm 212 while being collected with a condenser lens 210.

To the diaphragm 212, a motor 214 is mechanically coupled via a conveying mechanism such as an arm and a gear not shown. The motor 214 is a DC motor, for example, and drives under drive control by a driver 216. In order to render proper brightness to the image displayed on the display screen of the monitor 300, the diaphragm 212 is operated by the motor 214 to change the degree of opening. The light amount of the white light L radiated by the lamp 208 is restricted according to the degree of opening of the diaphragm 212. The reference of the brightness of the image considered proper is set/changed according to brightness control on the operation panel 218 by the operator. Note that the light control circuit that performs brightness control by controlling the driver 216 is a known circuit, and thus description thereof is omitted here.

The white light L having passed through the diaphragm 212 is collected to the incident end face of a light carrying bundle (LCB) 102 and input inside the LCB 102. The white light L input into the LCB 102 from its incident end face propagates through the LCB 102.

The white light L having propagated through the LCB 102 is radiated from the outgoing end face of the LCB 102 located at the tip of the electronic scope 100 to irradiate a body tissue in a body cavity via a light distribution lens 104. Return light from the body tissue irradiated with the white light L forms an optical image on a light receiving surface of a solid-state imaging device 108 via an objective lens 106.

The solid-state imaging device 108 is a single-plate color charge coupled device (CCD) image sensor having a Bayer pixel array. The solid-state imaging device 108 accumulates an optical image formed on each pixel of the light receiving surface as charge corresponding to the light amount, generates red (R), green (G), and blue (B) pixel data (shot image data), and outputs the generated data. Note that the solid-state imaging device 108 is not necessarily a CCD image sensor, but may be replaced with a complementary metal oxide semiconductor (CMOS) image sensor or any other kind of imaging device. The solid-state, imaging device 108 may also be one including a complementary filter.

A driver signal processing circuit 112 is provided in a connecting part of the electronic scope 100. Pixel data of the pixels obtained by imaging the body tissue irradiated with the white light L are input into the driver signal processing circuit 112 from the solid-state imaging device 108 every frame period. The driver signal processing circuit 112 executes processing, such as defective pixel correction, de-mosaicing, and correction processing unique to the solid-state imaging device 108, for the pixel data received from the solid-state imaging device 108, and outputs RGB format (or RAW format) pixel data to a signal processing circuit 220 of the processor 200. Note that in the following description, the “frame” may be replaced with the “field.”

The driver signal processing circuit 112 also accesses a memory 114 and reads unique information of the electronic scope 100. Examples of the unique information of the electronic scope 100 recorded in the memory 114 include the number of pixels and sensitivity of the solid-state imaging device 108, the operable frame rate, and the model number. The driver signal processing circuit 112 outputs the unique information read from the memory 114 to the system controller 202.

The system controller 202 performs various types of computation based on the unique information of the electronic scope 100, to generate control signals. Using the generated control signals, the system controller 202 controls the operations and timing of various circuits in the processor 200 so that suitable processing be performed for the electronic scope connected to the processor 200.

The timing controller 204 supplies clock pulses to the driver signal processing circuit 112 according to the timing control by the system controller 202. The driver signal processing circuit 112 controls the drive of the solid-state imaging device 108 at the timing synchronizing with the frame rate of an image processed on the processor 200 side according to the clock pulses supplied by the timing controller 204.

The signal processing circuit 220 provided in the processor 200 includes a matrix circuit 222, a YUV converting circuit 224, an edge enhancing circuit 226, and an output circuit 228.

The matrix circuit 222 executes matrix processing for the RGB format image data input from the driver signal processing circuit 112 every frame period, and outputs the processed data to the YUV converting circuit 224.

The YUV converting circuit 224 converts the matrix-processed pixel data (RGB format) input from the matrix circuit 222 to a YUV format, and outputs luminance signals (Y) and color-difference signals (U, V) obtained by the conversion to the edge enhancing circuit 226 and the output circuit 228, respectively.

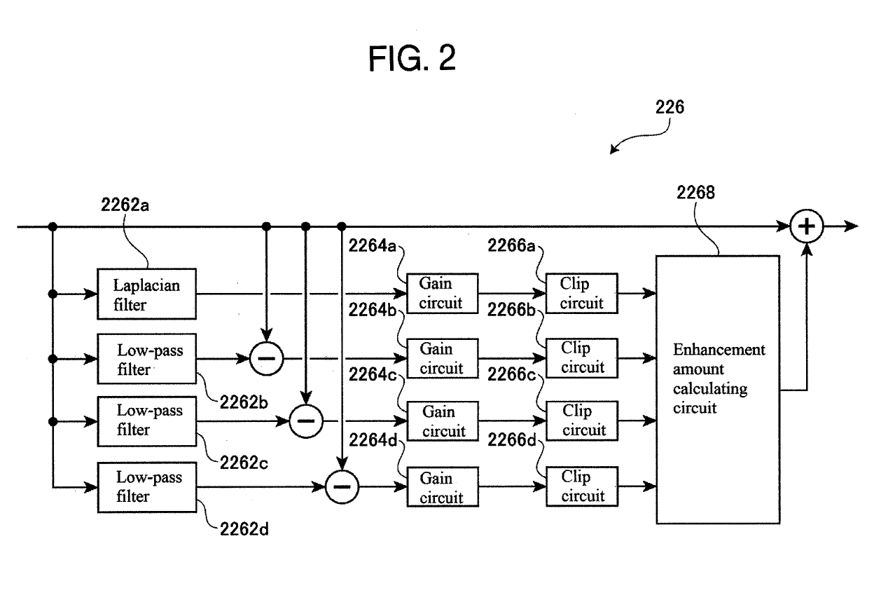

FIG. 2 is a block diagram showing a configuration of the edge enhancing circuit 226. As shown in FIG. 2, the edge enhancing circuit 226 has a Laplacian filter 2262a, low-pass filters 2262b, 2262c, and 2262d, gain circuits 2264a, 2264b, 2264c, and 2264d, clip circuits 2266a, 2266b, 2266c, and 2266d, and an enhancement amount calculating circuit 2268.

The Laplacian filter 2262a is filter-designed with a coefficient effective for edge detection (i.e., a coefficient suitable for detecting fine edges), and outputs a value obtained by multiplying, by the coefficient, a spatial second-order derivative in the luminance signal (Y) of an object pixel, as well as the luminance signals (Y) of its surrounding pixels, input from the YUV converting circuit 224 (i.e., of the original image). The resultant sharpened data of the object pixel is input into the gain circuit 2264a.

The low-pass filters 2262b, 2262c, and 2262d are filter-designed have 9 (3×3), 25 (5×5), and 49 (7×7) taps, respectively. The low-pass filters 2262b, 2262c, and 2262d have a filter design of averaging the luminance signal (Y) of the object pixel and the luminance signals (Y) of its surrounding pixels (i.e., all the filter coefficients in the filter have the same value) or a filter design according to the Gauss function (i.e., the filter coefficient is higher as the location is nearer to the center in the filter). Downstream of the low-pass filters 2262b, 2262c, and 2262d, the output value (non-sharp image data) of each low-pass filter is subtracted from the luminance signal (Y) of the object pixel of the original image. The resultant sharped data of the object pixel are input into the gain circuits 2264b, 2264c, and 2264d.

Note that the sharpened frequency components are higher by adopting the latter filter design (according to Gauss function) than adopting the former filter design (averaging). Also, using sharpened data obtained by subtracting the output value of a low-pass filter having a larger number of taps, lower frequency components are enhanced in the enhanced image, and the edge appears outstanding (thick). In other words, using sharpened data obtained by subtracting the output value of a low-pass filter having a smaller number of taps, higher frequency components are enhanced in the enhanced image, and the edge appears faint (thin).

While the Laplacian filter 2262a is especially effective for edge detection, the low-pass filters 2262b, 2262c, and 2262d are especially effective for sharpening of asperities in an image, and less generate noise in the enhanced image compared with the Laplacian filter 2262a. To state additionally, the frequency components of edges detected when using the Laplacian filter 2262a, the low-pass filters 2262b, 2262c, and 2262d are different from one another, and this means that the frequency components that can be enhanced with these filters are different from one another.

In the gain circuits 2264a, 2264b, 2264c, and 2264d, the sharpened data of the object pixel input from the upstream subtractors are gain-adjusted with gain values set in the respective gain circuits. The sharpened data of the object pixel gain-adjusted by the respective gain circuits are input into the clip circuits 2266a, 2266b, 2266c, and 2266d.

In this embodiment, a plurality of kinds of frequency enhancing modes (the low-frequency enhancing mode, the intermediate-frequency enhancing mode, and the high-frequency enhancing mode) are prepared. The operator can set the frequency enhancing mode appropriately by operating the operation panel 218. The gain values of the gain circuits are changed according to the set frequency enhancing mode. Otherwise, setting the gain values of the gain circuits may be made possible independently and directly by the operator operating the operation panel 218.

In the clip circuits 2266a, 2266b, 2266c, and 2266d, the sharpened data of the object pixel input from the gain circuits 2264a, 2264b, 2264c, and 2264d are clipped to values falling within a predetermined range, and output to the enhancement amount calculating circuit 2268. By the clipping by the clip circuits, the upper and lower limits of the sharpened data are regulated properly, whereby black rims and white rims in the image, called cats' eyes, are reduced.

In the enhancement amount calculating circuit 2268, the four lines of sharpened data input from the clip circuits are added up at a predetermined ratio (e.g., 0.25 0.25 0.25 0.25). Downstream of the enhancement amount calculating circuit 2268, the added-up sharpened data of the object pixel is added to the luminance signal (Y) of the object pixel of the original mage, and the resultant data is output to the output circuit 228.

The output circuit 228 converts the luminance signal (Y) input from the edge enhancing circuit 226 and the color-difference signals (U, V) input from the YUV converting circuit 224 to a predetermined video format signal. The output circuit 228, converting sequentially-input data of pixels to predetermined video format signals, outputs the converted signals to the monitor 300. Hence, an image in which the enhanced image obtained by enhancing specific frequency components in the body tissue has been superimposed on the normal color image is displayed on the display screen of the monitor 300.

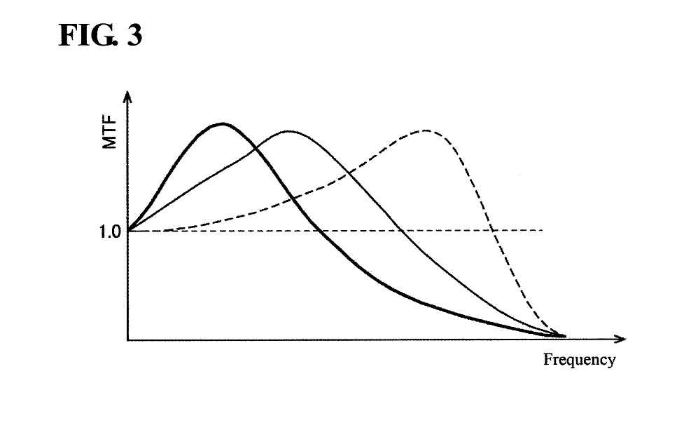

FIG. 3 shows the relationship between the frequency components and the MTF in the enhanced image output to the monitor 300. In FIG. 3, the y-axis represents the MTF (relative values with no unit of quantity) and the x-axis represents the frequency (relative values with no unit of quantity). In FIG. 3, also, the bold solid line represents the characteristics in the low-frequency enhancing mode, the thin solid line represents the characteristics in the intermediate-frequency enhancing mode, and the broken line represents the characteristics in the high-frequency enhancing mode.

During the low-frequency enhancing mode, for example, the gain value of a gain circuit located downstream of a low-pass filter having a large number of taps is set at a relatively high value, and the the gain value of the gain circuit 2264a located downstream of the Laplacian filter 2262a is set at a low value. As an example, the highest gain value is set for the gain circuit 2264d located downstream of the low-pass filter 2262d having 49 taps, and subsequently higher gain values are set for the gain circuits 2264c, 2264b, and 2264a in this order.

As described above, during the low-frequency enhancing mode, the ratio of the data obtained by detecting edges of low-frequency components (sharpened data obtained by subtracting the output value of a low-pass filter having a large number of taps) becomes relatively high, and the ratio of the data obtained by detecting fine edges (sharpened data from the Laplacian filter 2262a) becomes relatively low. Therefore, as shown in FIG. 3, in the enhanced image during the low-frequency enhancing mode. the MTF of comparatively low frequency components becomes high. In other words, the enhanced image during the low-frequency enhancing mode is one in which comparatively low frequency components have been enhanced.

During the intermediate-frequency enhancing mode, compared with the low-frequency enhancing mode, the gain value of a gain circuit located downstream of a low-pass filter having a small number of taps is set at a high value, and the the gain value of the gain circuit 2264a located downstream of the Laplacian filter 2262a is set at a high value. As an example, the gain values of all the gain circuits 2264a, 2264b, 2264c, and 2264d are set at the same value.

As described above, during the intermediate-frequency enhancing mode, compared with the low-frequency enhancing mode, the ratio of the data obtained by detecting edges of high-frequency components (sharpened data from the Laplacian filter 2262a and sharpened data obtained. by subtracting the output value of a low-pass filter having a small number of taps) becomes high. Therefore, as shown in FIG. 3, in the enhanced image during the intermediate-frequency enhancing mode, the peak of the MTF shifts to the high-frequency side from that during the low-frequency enhancing mode. In other words, the enhanced image during the intermediate-frequency enhancing mode is one in which high frequency components have been enhanced by increasing the ratio of filters that detect edges of high-frequency components, compared with that during the low-frequency enhancing mode.

During the high-frequency enhancing mode, compared with the intermediate-frequency enhancing mode, the gain value of a gain circuit located downstream of a low-pass filter having a small number of taps is set at a high value, and the gain value of the gain circuit 2264a located downstream of the Laplacian filter 2262a is set at a high value. As an example, the highest gain value is set for the gain circuit 2264a and subsequently higher gain values are set for the gain circuits 2264b, 2264c, and 2264d in this order.

As described above, during the high-frequency enhancing mode, compared with the intermediate-frequency enhancing mode, the ratio of the data obtained by detecting edges of high-frequency components (sharpened data from the Laplacian filter 2262a and sharpened data obtained by subtracting the output value of a low-pass filter having a small number of taps) becomes high. Therefore, as shown in FIG. 3, in the enhanced image during the high-frequency enhancing mode, the peak of the MTF shifts to the high-frequency side from that during the intermediate-frequency enhancing mode. hi other words, the enhanced image during the high-frequency enhancing mode is one in which high frequency components have been enhanced by increasing the ratio of filters that detect edges of high-frequency components, compared with that during the intermediate-frequency enhancing mode.

For example, the low-frequency enhancing mode is suitable for enhancement of shot images of the large intestine having many large blood vessels and asperities, etc., and the high-frequency enhancing mode is suitable for enhancement of shot images of the esophagus and stomach on the surfaces of which microscopic blood vessels are located, etc.

As described above, the edge enhancing circuit 226 according to this embodiment is provided with a plurality of filters for enhancing different frequency components, and respective sharpened data subjected to filtering by the filters are added up at a predetermined ratio. The frequency components to be enhanced in the image vary depending on the level ratio of the sharpened data before adding-up. By setting the frequency enhancing mode in accordance with the site in the body cavity (i.e., by adjusting the level ratio of the sharpened data), the operator can display an enhanced image of the site corresponding to the mode.

While an illustrative embodiment of the present invention has been described hitherto, the present invention is not limited to the foregoing embodiment, but various modifications are possible within the scope of the technical ideas of the present invention. For example, appropriate combinations of the embodiment illustratively expressed herein and obvious embodiments, etc. are also included in the embodiments of the present invention.

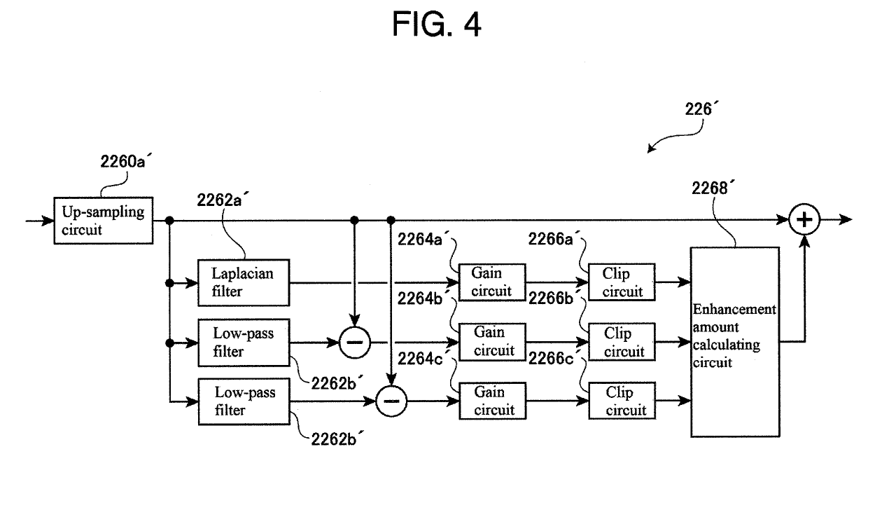

FIG. 4 is a block diagram of a configuration of an edge enhancing circuit 226′ according to another embodiment of the present invention. As shown in FIG. 4, the edge enhancing circuit 226′ has an up-sampling circuit 2260′, a Laplacian filter 2262a′, low-pass filters 2262b′ and 2262c′, gain circuits 2264a° , 2264b′, and 2264c′, clip circuits 2266a′, 2266b, and 2266c°, and an enhancement amount calculating circuit 2268′.

The up-sampling circuit 2260′ increases the granularity (i.e., frequency or resolution) of the original image, for the signals of the pixels of the original image input from the YUV converting circuit 224, using a known method such as Laplacian pyramid method.

The luminance signal (Y) of the object pixel, as well as the luminance signals (Y) of its surrounding pixels, of the original. image the granularity of which has been increased by the up-sampling circuit 2260′ are input into the Laplacian filter 2262a′, the low-pass filters 2262b′ and 2262c°. The subsequent processing is the same as that for the edge enhancing circuit 226 shown in FIG. 2.

As described above, according to this embodiment, the granularity of the original image is increased by the up-sampling circuit 2260′ before the luminance signals (Y) of the original image are input into the Laplacian filter 2262a′, the low-pass filters 2262b′ and 2262c′. Therefore, even when the number of pixels of the solid-state imaging device 108 is small, it is possible to generate an enhanced image in which high-frequency components have been enhanced. Also, to realize a configuration suitable for enhancement of high-frequency components, only low-pass filters small in the number of taps (9 (3×3) and 25 (5×5) in this example) are provided.

1. An image processing device comprising:
a plurality of sharpening circuits for enhancing different frequency components for an original image;
an adjuster that adjusts a signal level ratio of an image sharpened by each of the plurality of sharpening circuits; and
a generator that generates an enhanced image by adding up signals of images subjected to the signal level ratio adjustment at a predetermined ratio.

2. The image processing device of claim 1, comprising a granulator that increases granularity of the original image upstream of the plurality of sharpening circuits.

3. The image processing device of claim 1, comprising an aggregator that adds up a signal of the original image and a signal of the enhanced image generated by the generator.

4. The image processing device of claim 1, comprising a clipper that clips the signal of each image subjected to the signal level ratio adjustment by the adjuster.

5. The image processing device of claim 1, wherein the plurality of sharpening circuits include
a set of a low-pass filter and a subtraction circuit that subtracts an output signal of the low-pass filter from the signal of the original image, and
a Laplacian filter.

6. An electronic endoscope system comprising:
an electronic scope; and
the image processing device of claim 1 that processes image data shot in the electronic scope as the original image.
